(19) United States
(12) Patent Application Publication
(10) Pub. No.: US 2008/0262347 A1
(43) Pub. Date: Oct. 23, 2008

(54) METHOD AND APPARATUS FOR MONITORING INTEGRITY OF AN IMPLANTED DEVICE

(76) Inventors: Geoffrey Batchelder, Chandler, AZ (US); Jeffrey H. Nycz, Collierville, TN (US); Carlos E. Gil, Collierville, TN (US); Jeetendra Bharadwaj, Memphis, TN (US)

Correspondence Address: HAYNES AND BOONE, LLP, 901 Main Street, Suite 3100, Dallas, TX, 75202, US

(21) Appl. No.: 11/738,036

(22) Filed: Apr. 20, 2007

(51) Int. Cl. A61B 8/00

(52) U.S. Cl. 600/437

(57) ABSTRACT

A system for detecting the integrity of a previously implanted prosthetic device may include a detector including a sensor configured to detect signals from the implant and securing means for securing the sensor in a sensing position relative to the implantable device. The system also may include a power source configured to provide power to the sensor and a computation means for processing data communicated by the detector and for displaying the processed data for diagnosing.

### TECHNICAL FIELD

This invention relates in general to a method and apparatus for monitoring the integrity of a previously implanted prosthetic device.

### BACKGROUND

Implanted prosthetic devices provide critical support to bone structures in the body. In some instances, these devices may be implanted and cemented in place against the bone they support. In other instances, they may be secured in place by osteointegration. Yet sometimes, over time, the implant to bone interface begins to degrade, causing a loosening of the implant relative to the bone structure. This may occur for a wide variety of reasons including, for example, failure of or damage to the implant, failure of or damage to the bone structure adjacent the implant, degradation of the cement, poor tissue healing, the deterioration of the function and/or shape of the interfacing bone structure after the surgical correction, and/or other patient-related factors.

Because of this, physicians may desire to monitor the physical mechanical integrity of the implant and the adjacent bone structure through periodic follow-up visits. One way of doing so includes taking Radiographs and visually identifying irregularities such as dislocation or movement of the implant relative to the bone structure. However, using this method, irregularities may not be visually identifiable until after the displacement has grown so large that major revision surgery is required. For example, some implant displacement may not be visually identifiable on an x-ray until the implant has displaced up to or beyond 2 mm. Once such large displacement has occurred, simple procedures often cannot rectify the displacement. Accordingly, the implant may typically be replaced and/or degraded bone structure may typically be removed. These major surgeries may result in high levels of trauma and distress to the patient.

What is needed is a system and method that allows simple monitoring of the integrity of the implanted device and the interfacing bone structure.

### SUMMARY

In one exemplary aspect, the present disclosure is directed to a detector including a pliable material configured for placement on a patient adjacent to a previously implanted medical device and also including a sensor attached to the material. The sensor may be configured to detect signals from the implant. In some aspects, the sensor may be one of: an acoustical sensor configured to detect an acoustical signal generated at the implant and an sonic transducer configured to detect reflected sonic waves, such as for example, ultrasonic waves. In some exemplary aspects, the sensor may be a plurality of sensors disposed in an array and configured to detect signals from more than one location on the implant. In some exemplary aspects, the detector may include a wireless transmitter associated with the pliable material.

In another exemplary aspect, the present disclosure is directed to a system for detecting the integrity of a previously implanted prosthetic device. The system may include a detector including a sensor configured to detect signals from the implant and securing means for securing the sensor in a sensing position relative to the implantable device. The system also may include a power source configured to provide power to the sensor and a computation means for processing data communicated by the detector and for displaying the processed data for diagnosing.

In another exemplary aspect, the present disclosure is directed to a method including disposing a sensor outside a patient's body for detecting a signal generated at an implanted prosthetic device and detecting with the sensor the signal generated around the implanted prosthetic device. The method also may include determining, based on the detected signal, the integrity of an interface between the previously implanted device and adjacent bone structure. In some aspects, the sensor may be an acoustically sensitive sensor, and wherein detecting with the sensor includes detecting acoustics generated at the implant.

In one exemplary aspect, the present disclosure is directed to a method including providing a sensor for disposal outside the patient's body, the sensor being an acoustically sensitive sensor for detecting an acoustical signal generated at an implanted prosthetic device. Computer program product may be provided for formatting a computation means for determining, based on the detected signal, the integrity of an interface between the previously implanted device and adjacent bone structure.

### BRIEF DESCRIPTION OF THE DRAWINGS

FIG. 1 is an illustration including a partial block diagram of an exemplary system for monitoring the integrity of a previously placed prosthetic device.

FIGS. 2A and 2B are illustrations of an exemplary detector as an implant integrity wrap.

FIG. 3 is an illustration of an exemplary detector disposed on a patient to monitor a previously placed prosthetic hip replacement device.

FIG. 4 is an illustration of an exemplary sensor that may form a part of a detector.

FIG. 5 is an illustration of another exemplary detector.

FIG. 6 is a flow chart showing exemplary method of monitoring the integrity of a previously implanted device.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

This disclosure is directed to devices, methods, and systems that provide indications of the integrity of implanted prosthetic devices. Conventional systems may employ an X-ray for visual analysis of an implanted device. If displacement is detected in the X-ray, then an evaluating physician concludes that the implant is loose or detached from the bone or cement. Subsequent surgery, such as a major revision surgery, may be required to relocate or replace the displaced device.

In contrast, the devices, methods, and systems disclosed herein may allow early detection of any loosening of the device. Accordingly, any treatment to repair the device also may be accomplished early, potentially avoiding the need for a replacement device or for a major revision surgery. Instead, because of the early detection, minimally invasive procedures may be used to re-cement or otherwise re-secure the implant in its place against the adjacent bone. Using a minor revision surgery, such as a minimally invasive surgery may be less traumatic for the patient, be less painful, and may allow a faster recovery rate. Furthermore, in some embodiments, the devices, methods, and systems disclosed herein rely upon data, such as acoustical signals, rather than visual images to monitor the implant integrity.

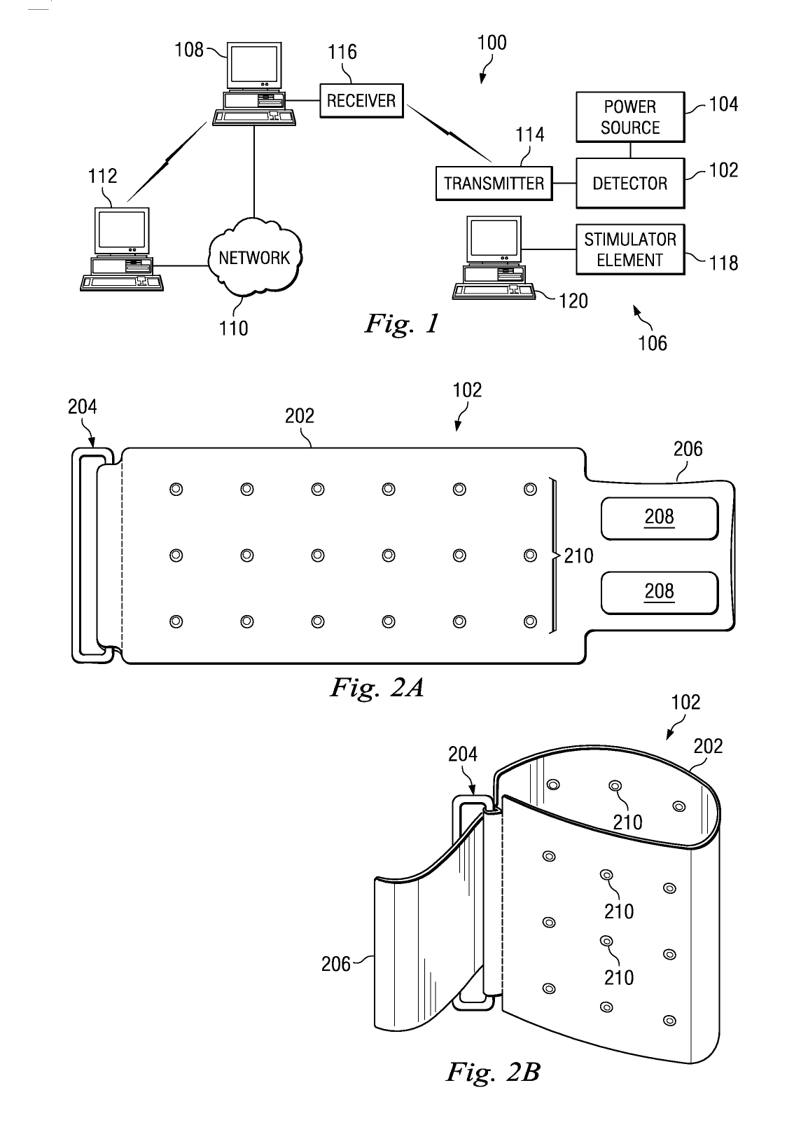

One exemplary system for monitoring the integrity of a previously placed implant is shown in FIG. 1 and is referenced herein by the reference numeral 100. The system 100, in this exemplary embodiment, includes a detector 102, a power source 104, an optional stimulator 106, and a processing system 108. The system 100 also optionally may include a network such as the internet 110, in communication with the processing system 108 and also may include one or more additional processing systems 112 capable of communicating over the network, or alternatively, directly connected with the processing system 108. The different components of the exemplary system 100 are described further with reference to other embodiments of the invention.

The detector 102 is configured to detect signals, waves, or acoustical data indicative of the integrity of the implant. In this embodiment, the detector 102 is configured to non-invasively detect the data. One exemplary detector 102 is shown in FIGS. 2A and 2B. In this exemplary embodiment, the detector 102 is a band or wrap formed of a pliable material configured to extend around a portion of the body containing the previously implanted prosthetic device, for example, the detector may be sized to fit around an upper thigh, arm, torso, knee or other body part of a human patient where an implant may be present. In some exemplary embodiments, the detector 102 is formed of a pliable cloth-like material configured to conform to a body shape, such as around a limb. In some embodiments, it may be a non-stretchable material.

In the exemplary embodiment shown, the detector 102 includes a sensing body portion 202, a buckle portion 204, and a tongue portion 206. The buckle portion 204 is attached to one end of the body portion 202 and the tongue portion 206 extends from the other end. In this exemplary embodiment, the tongue portion 206 also includes one or more strips of either a hook or a loop of a hook and loop fastener 208.

As shown in FIG. 2B, the tongue portion 206 may be fit through the buckle portion 204 to form a tubular shape wrappable about a body. The hook and loop fastener 208 may wrap around and connect to corresponding hook and loop fasteners (not shown) on the body portion 202 of a back side of the tongue portion 206. It should be noted that the buckle and tongue arrangement discussed herein is one exemplary system for wrapping the detector around a patient's body. However, any suitable system could be used, including a single wrap with hook and loop fasteners without a buckle portion, a body portion having a separate wrapping component, such as a tie, that secures the body portion on the patient, a ribbon-type wrap that can be tied, a body portion having a buckle, among any others.

The detector 102 includes one or more associated sensors 210 for detecting the data indicative of the integrity of the implant. In the embodiment shown, the sensors 210 are disposed in the body portion 202 and configured to be applied against the patient, such as on the patient's skin, to detect acoustical signals generated at the implant to bone interface. In some embodiments, the sensors 210 may be attached to or embedded into the body portion 202. In other embodiments, the sensors 210 are attached to a band held in place by the body portion 202. In FIGS. 2A and 2B, the sensors 210 form an array. In different embodiments, the array may be linear, curved, or multi-dimensional. In some exemplary embodiments, only a single sensor is employed.

The sensors 210 may be acoustical sensors capable of detecting acoustical waves emitted at the implant to bone interface within the patient's body, and carried through the patient's body to the sensors, that may be disposed at the skin. In some exemplary embodiments, the sensors 210 are piezoceramic/piezoelectric sensors. For example, these may include pressure sensors and Acoustic Emission transducers. In other exemplary embodiments, the sensors may be accelerometers. These different types of sensors have different frequency response curves and constructions. For example, the pressure sensor may have sensitivity at low frequencies, such as for example, in the range of 10 Hz to 50 kHz, while the Acoustic Emission transducer may operate in the range of, for example about 12 kHz to 900 kHz. In some exemplary embodiments, the sensors utilize a frequency spectrum range from 200 Hz to 500 kHz. In some embodiments, different sensors 210 of the detector 102 include various frequency response characteristics.

In some exemplary embodiments, the sensors 210 are ultrasound transducers. Other sensors also may be used. In some embodiments, the sensors 210 detect loosening of the implant relative to the bone or cement material through impedance, acoustics, ultrasound, vibration, or other physical parameter variations.

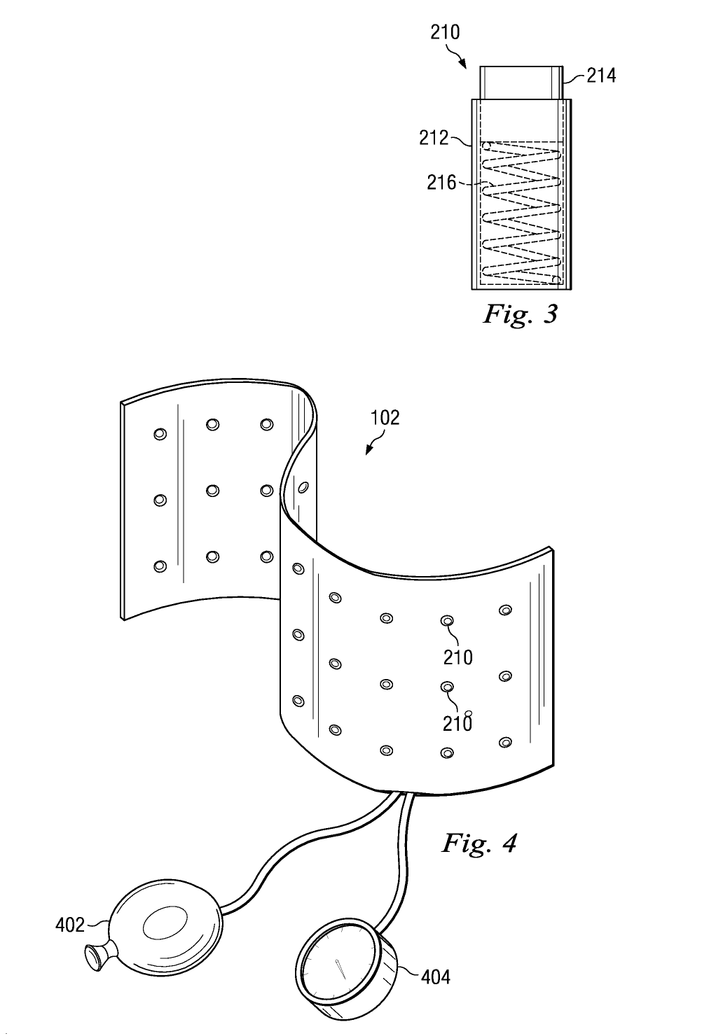

In some embodiments, in order to properly detect data generated at the implanted device, the sensors 210 must be maintained in direct contact with the skin. One example of a sensor 210 that maintains the sensor in direct contact with the skin is shown in FIG. 3. In this embodiment, the sensor 210 includes a housing as a cylindrical tube 212 that receives a sensing head 214 and a physical or mechanical biasing element, such as a spring 216. The spring 216 is disposed behind the sensing head 214 within the tube 212, biasing the sensing head 214 outwardly out of the tube 212. In one embodiment, each sensor 210 in FIG. 2 includes a biased sensing head 214 configured to maintain contact with the skin with a sufficient force to enable suitable reception of the acoustical signals. In these embodiments, the tube 212 may be fixed to the body portion 202 of the detector 102 at spaced locations, such that the sensors 210 may be aligned in rows and columns forming an array of sensors. The tubes 112 may be attached to the body 202 using an adhesive, may be sewn to the body, may be welded or melted into the body 202, or otherwise attached. In addition to sensors 210 having tubes and biasing elements, other methods and systems could be used to maintain the sensor in contact with the skin. In some exemplary embodiments, the body portion 202 of the detector 102 itself, without the use of separate biasing elements suitably maintains the sensors 210 in contact with the skin.

Yet another example of the detector is shown in FIG. 4. In this exemplary embodiment, the detector 102 may include an inflatable portion, such as the body portion 210. The inflatable portion may inflate in a manner similar to that of a blood pressure measuring cuff. Accordingly, in such an example, the detector 102 may include a squeezable bulb or bladder 402 for inflating the detector 102 and a pressure gauge 404 for monitoring the pressure about the limb. Inflating the detector 102 by pumping the bladder 402 may replace or supplement a biasing element to suitably place the sensors 210 in a position against the skin for detection of acoustical waves. Such a system also may provide consistency and repeatability by allowing a physician to secure the sensors against the skin with a repeatable pressure.

Returning to FIG. 1, the detector 102 may include a means for communicating data indicative of the received signals to the processing system 108. In the exemplary embodiment in FIG. 1, this is done wirelessly, such as, for example, by telemetry, by RF transmission, by infrared transmission, or other wireless information transfer system. Accordingly, the detector 102 also may include a transmitter 114 operable to transfer information from the detector to the processing system 108. In some embodiments, the detector 102 may include a hardware interface such as a USB or SCSI adapter to transfer data collected to a database, such as may be included in the processing system 108.

In some embodiments, the detector 102 is connected by a wire connection to the processing system 108. Further, in some exemplary embodiments, the processing systems 108 is a miniature processing system that may be attached to and carried on the detector 102.

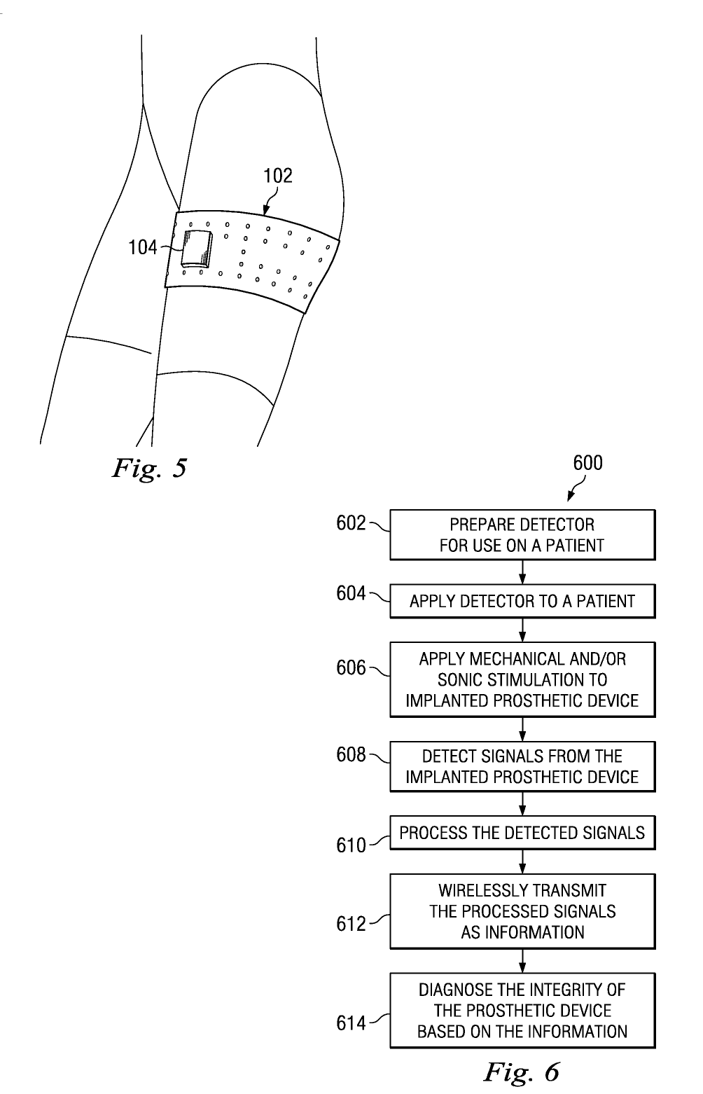

The power source 104 from FIG. 1 may be associated with the detector 102 in any known manner to provide energy to and power the sensors 210 in the detector 102. In some embodiments, the power source 104 is a battery pack associated with the detector, such as for example, directly attached to the detector 102. One example of this is shown in FIG. 5, which shows a detector 102 with an attached battery pack power source 104 disposed about a patient's thigh for monitoring of a previously implanted acetabular cup. Here, the detector 102 may be applied over the greater trochanter of the femur and iliac crest of the pelvis. The attached power source 104 makes the detector 102 mobile, as it is not connected by wires to an external power source. It still may be removable from the detector 102 for cleaning or simple battery replacement. Although in some embodiments, the power source 104 is a battery pack that may be carried by the patient, in other embodiments the power source 104 is connected by a wire to an electrical outlet or other source. In some embodiments, the power source 104 is also the processing system 108. For example, the detector 102 may connect to the processing system 108 through a USB cable that provides power to the detector 102, but also communicates data from the detector 102 to the processing system 108.

The processing system 108 may be a computer system having a processor and memory configured to receive and process data sent from the sensors 210 of the detector 102. The processing system 108 may include input devices, such as a keyboard, mouse, among others, and may include an output device such as a display or other output device. In some examples, when the detector 210 includes a wireless transmitter, the processing system 108 may include a wireless interface, such as a receiver capable of receiving broadcast data or information indicative of the data gathered by the sensors 210. Some examples may be by telemetry, by RF transmission, by infrared transmission, among others. In other embodiments, when the detector 102 includes a wired hardware interface, the processing system 108 may include corresponding hardware to communicate with the detector 102. For example, the processing system 108 may include a hardware interface such as a USB or SCSI adapter to receive data collected by the detector 102.

The memory and processor of the processing system 108 may be configured in a manner to receive data from the sensors 210 and process the data into information indicative of the integrity of the implant. In some embodiments, the memory and processor are conventional components, but include computer executable programs that evaluate the data from the sensors and output the data to a display that may be interpreted by an operator, such as a physician or technician. The data detected by and sent from the sensors 210 can be analyzed singularly or in aggregate. In some exemplary embodiments, the processing system 108 and the detector 102 may include noise reduction processes that may assist in extracting signals from a noisy background environment, or alternatively, in some embodiments, the sensors 210 and the detector 102 may include such noise reduction capabilities. The system 108 may analyze signals using a variety of signal analysis methods. One example of signal analysis method employs Acoustic Emission Analysis, a technique used in industry that is designed to detect the release of acoustic energy from members under load as defects yield under stain. In other embodiments, the processing system 108 employs Fourier analysis, such as Fourier spectral analysis. Phase analysis, wavelet analysis, and various forms of signal filtering analysis also may be used.

As described above, in some exemplary embodiments, the processing system 108 may be attached to and carried on the detector 102. For example, the processing system 108 may include a processing system, such as for example, a PALM type or other processing system, capable of collecting data from the sensors 210, performing some level of processing, and then transmitting information to the processing system 112. Accordingly, in these embodiments, the processing system 108 may be similar to a handheld device, a PDA, or other mobile system.

The processing system 108 may be in communication with the network 110. The network 110 may be any wired or wireless LAN or WAN, including the Internet and may allow transfer of the processed data from the processing system 108 to a separate processing system, such as the processing system 112. In some embodiments, the data is transferred over the network as a part of an email message, while in others, the information on the processing system 108 is accessed by requests from the user computer 112. Also, as shown in FIG. 1, in some embodiments, the processing system 112 is directly connected to or in communication with the processing system 108. Any of the connections described herein may be wired or wireless connections.

In some exemplary embodiments, the processing system 108 is disposed apart from the detector, such that data collected by the detector 102 is transmitted to and received by the processing system 108. For example, the detector 102 may be in an examining room and the processing system 108 may be in a separate room, such as a medical provider's office. In some embodiments, the processing system 108 may be located to receive data from multiple detectors that may be disposed in multiple locations, such as in multiple examining rooms.

The stimulator 106 is an optional component for applying a mechanical force of a load to the implanted prosthetic device within the patient. It may include a stimulating element 118 and a processing system 120. The stimulator 106 may be a stand-alone system as shown, or alternatively, may be in communication with the processing system 108. In some embodiments, the processing system 120 is the same processing system as processing system 108.

In some exemplary embodiments, the stimulating element 118 is a vibrator configured to impart resonant or non-resonant mechanical vibration of a specific spectral content or other physical movement to a point on the bone region containing the implantable device. For example, in some exemplary embodiments, the stimulating element 118 includes a vibrating head configured to be placed on the skin to impart motion to the implantable device relative to the adjacent bone structure. This actively imparts energy, through a vibration mechanism to stimulate movement of the bone-implant assembly.

In other exemplary embodiments, the stimulating element 118 is a sonic or ultrasonic transducer to be applied to the tissue over the region of the implanted bone for the purpose of transmitting sonic energy to the implant to bone interface. In some embodiments, any reflected waves may be detected by the detector 102. In some exemplary embodiments, the same transducer may operate as the detector, and in other exemplary embodiments, the detector may be a separate transducer.

The processing system 120 may be configured to control the stimulating element. For example, it may control the frequency of the vibrations or the frequency of the emitted ultrasonic waves. In some exemplary embodiments however, the frequencies are not closely controlled, but the stimulator 106 may be an off-the-shelf vibrating or wave emitting device, such as a conventional electrical massaging device. In these instances, the processing system 120 may be merely a power source and a switch that operates the stimulator 106, which may be as basic as a single speed, non-adjustable electric motor in a housing.

An exemplary method of using the system 100 for monitoring the integrity of the implant is described with reference to FIG. 6, and referred to by the reference numeral 600. The method provides a non-invasive method that can be readily applied in a clinical setting. The sensors 210 of the detector 102 may be applied to the patient's skin and held in place to provide suitable contact with skin for efficient acoustical signal transmission without causing discomfort to the patient. The method also may allow the patient to be able to move in a variety of ways during the testing in order to apply mechanical loading onto the implant if desired. Local anesthetic may be used to allow the movements to proceed, if desired.

The method begins at a step 602, where the detector 102 is prepared for placement on the patient. In some embodiments, the detector 102 is applied against the skin of the patient. Preparing the detector may include placing a couplant such as, for example, an ultrasonic gel on the detector 102 or on the patient to aid in the transfer of the signals from the patient to the sensors 210.

Once the couplant is applied, the detector 102 may be placed on the patient, as at step 604. In some embodiments, the detector 102 is a band or wrap that extends about a portion of the patient's body, such as for example, about at least a portion of a limb. The wrap may be sized to fit about a substantial portion of a body having the implant disposed therein. As described above, the sensors 210 may be disposed in the an array formed of a plurality of sensors or may include only a single sensor. In some embodiments employing a plurality of sensors 210, the detector 102 may be configured to detect or collect acoustic signals emanating from two or more different locations along the implant to bone interface. This occurs because the sensors 210 may be spaced apart in different locations of the detector 102. In some exemplary embodiments, applying the detector 102 may include orienting or manipulating the detector so that associated sensors are in a position to properly detect acoustics generated at the implant. This may include securing the sensors against the patient, such as against the patient's skin by for example, tightening the detector or by inflating the detector. In one example of applying the detector to a patient to detect monitor the integrity of the implant, the detector is applied to a evaluate a hip joint. In this example, the detector 102 may be applied over the greater trochanter of the femur and iliac crest of the pelvis.

At step 606, mechanical stimulation and/or sonic stimulation optionally may be applied to the implant. The mechanical stimulation may be applied as static loads (such as without movement) or dynamic loads (such as to impart or impact movement). Some loads may be relatively large loads, as when the implant is a hip replacement prosthesis and the patient is told to move the hip by walking, while other loads may be relatively small such as when vibrations are applied to create slight movement in the area around the implant. In some examples, the mechanical stimulation is applied by the patient's own weight. For example, when the implant is an acetabular cup, the patient may be instructed to raise or swing his leg, to walk across the room, or to move from a sitting position to a standing position, thereby mechanically stimulating, through loading, the implant. Other examples may include mechanically stimulating the implant to bone interface under cyclical loads, as may occur on a stationary bicycle, a stair stepper, or a tread mill. Other loadings are possible, including impact loadings and application of compressive or tensile loads.

In some exemplary embodiments, the loads may be applied through the stimulator 106. In these embodiments, the stimulator 106 may be disposed on the patient, for example, to impart vibrations or sonic energy to the implant to bone interface to stimulate some level of movement of the bone-implant assembly. When the loads are vibrational loads, they may include, for example, resonant or non-resonant mechanical vibrations of a specific spectral content to a point on the bone region containing the implanted prosthesis through the overlying tissues and apply acoustic sensors over locations of interest on the bone to measure, through the overlying tissues, the acoustic signals spectra generated by the implant motion within the bone to implant attachment region. In some embodiments, the stimulator may be a sonic or ultrasonic transducer and applying loads may include transmitting sonic energy to the implant to bone interface. Other methods and systems also may be used to impart loads on the interface. Accordingly, in this embodiment, the stimulator actively induces some level of movement at the bone-implant interface. It should be noted that in some exemplary embodiments, more than one of the loading, the vibrations, and the sonic energy are used simultaneously in an hybridized matter to stimulate the bone-implant interface.

Applying the loads may initiate relative movement between the implanted device and the adjacent bone structure. Accordingly, by simultaneously imparting or impacting loads and monitoring the implant integrity, signals may be detected that indicate separation of the implant from the bone/cement. For example, acoustic signals may be generated as the implant moves against the bone/cement. In some examples, debris from the implant or the bone/cement may slide along an articulating surface of the implant, providing distinctive acoustic signals that the implant is loosening. In other examples, the separation of the implant from the bone/cement may impart distinctive signals. Other conditions also may impart distinctive signals.

At a step 608, the detector detects waves or acoustical data as signals from the implant indicative of the integrity of the implant and the implant to bone/cement interface. The signals may be generated at the implant and bone or cement or alternatively, reflected by the implant and bone or cement. In some examples, the signals are acoustical signals generated at the implant to bone/cement interface. For example, if the implant begins to separate from the adjacent bone/cement structure, signals generated by or returning from the implant to bone interface may have signatures or patterns that indicate void formation on the bone/cement or an unconstrained interface.

In one exemplary embodiment, the detecting the signals at step 608 includes passively listening with sensors, such as acoustic sensors, for acoustical emission signals generated by the bone/cement to implant interface region as mechanical loads are applied to the implanted structure. Some of the signal contents that can be measured are industry standard acoustic emission parameters and the actual time evolution of the bone acoustic pressure waves as they impinge upon the sensor. In some embodiments, no external loading occurs while detecting the signals, while in other embodiments, static or dynamic loads may be applied during sensing, as described in step 606. The frequency spectral content of the acoustical waves sensed may be variable and may be dependent upon the exact mechanism generating the emissions. Nevertheless, the basis of the method is that implants at different degrees of looseness should produce distinctive signal patterns.

In embodiments where the stimulator 106 is a transducer, such as a sonic or ultrasonic transducer, the detecting at step 608 may include detecting sonic or ultrasonic waves reflected from the bone/cement-implant interface. In such embodiments, the detector 102 may comprise the emitting transducer of the stimulator or a second transducer or a plurality of transducers placed over a location on the patient to sense the waves reflected from the bone-implant regions. In some embodiments, phased arrays, focused sonic or ultrasonic, and high intensity focused ultrasound may be employed. Others also are contemplated.

In embodiments where the processing system 108 is separate from the detector 102, the data may be transferred to the processing system 108. This may be done through a wire connection, a wireless connection, or physical transfer, such as using a removable storage medium, such as for example, a removable floppy disc or flash drive. In some exemplary embodiments, as shown in FIG. 1, the detector 102 and the processing system 108 communicate via a transmitter and receiver system. This may be a telemetry system, an RF transmission, an infrared transmission, among any other suitable transmission types. As described above, in some embodiments, the processing system 108 is on the detector 102. Thus, the data transferred can be raw or reduced data.

At a step 610, the detected signals are processed or interpreted to create meaningful or readable data that may be indicative of the integrity of the implant. Processing may be accomplished by the processing system 108 and may incorporate software or hardware processing, including filtering to obtain meaningful information from the signals.

Processing also may include analyzing the data or information for specific attributes. These attributes may be determined by empirical means through clinical testing. Clinical specimens that show various degrees of detachment of the prosthesis from and also show discernable tissue morphology of the associated bone may be evaluated and the acoustic data collected may be assessed for specific amplitude and frequency relationships that are representative of the implant's looseness status.

Processing in step 610 may include comparing the data or information obtained at step 508 to the previously obtained empirical data to identify representative amplitudes and frequencies that indicate the integrity of the implant.

In some embodiments, the processing may include comparing the collected data or information to data or information for the patient that may have been previously collected and stored. Variations in signals between the previous data and the current data may be indicative of changes in the integrity of the implant to bone or implant to cement interface.

In other embodiments, the processing may include comparing the collected data or information to stored data taken from a group or class of reference cohorts. For example, the collected data may be compared to stored data taken from a class of persons with whom the patient fits. For example, the class of persons may be assigned based on, for example, age, gender, BMI, height, weight, bone density, among other characteristics. Thus, comparing the patient's data or information to the relevant class may provide a more accurate indication of the implant's looseness status.

In some embodiments as shown in step 612, once this information is obtained (or alternatively, in its raw, unprocessed form), it may be transmitted from the processing system 108 to another computer station or processing system such as processing system 112 in FIG. 1. This may occur by direct transmission or over a network. The processing system 112 may be placed for access by a care provider in another location either in the same building or elsewhere. For example, the information may be wirelessly transmitted from a clinic conducting the test to a treating physician's office, such as a surgeon's office, for his or her review. In some embodiments, this may be done through wireless telemetry. Also, in some embodiments, the processing at step 610 may occur after transmitting the information. Although shown at step 612 as a wireless transmission, transmission of the information may be over any wired or wireless network, such as a LAN or WAN, including the Internet. In some embodiments, the data is transferred over the network as a part of an email message, while in others, the information on the processing system 108 is accessed by requests from the user computer 112.

Once received by the care provider, the information and data may be used to diagnosis the integrity of the implant in the patient based on the data received, as indicated at step 614.

It should be noted that the selected embodiments are intended to include monitoring the integrity of a bone to implant interface as well as a cement to implant interface. Further, the following claims, reciting an interface between the device and bone structure are intended to be understood to include within their scope situations where cement is applied between the device and bone structure. Although several selected embodiments have been illustrated and described in detail, it will be understood that they are exemplary, and that a variety of substitutions and alterations are possible without departing from the spirit and scope of the present invention, as defined by the following claims.

We claim:

1. A detector, comprising:
a pliable material configured for placement on a patient adjacent a previously implanted medical device;
a sensor attached to the material, the sensor being configured to detect signals from the implant.

2. The detector of claim 1, wherein the sensor is one of:
an acoustical sensor configured to detect an acoustical signal generated at the implant; and
an sonic transducer configured to detect reflected sonic waves.

3. The detector of claim 1, wherein the sensor is a plurality of sensors disposed in an array and configured to detect signals from more than one location on the implant.

4. The detector of claim 1, wherein the pliable material includes a hook and loop fastener for securing the pliable material about a portion of the patient.

5. The detector of claim 1, wherein the pliable material is inflatable.

6. The detector of claim 1, further comprising:
an inflation bulb operably connected to the pliable material; and
a pressure gauge configured to detect pressure in the pliable material.

7. The detector of claim 1, wherein the pliable material is a non-stretchable material.

8. The detector of claim 1, wherein the sensor includes a housing operably associated with the pliable material.

9. The detector of claim 8, wherein the housing includes an inner space having a biasing device disposed therein, the biasing device being configured to bias a sensor head out of the housing.

10. The detector of claim 1, further including a power pack associated with the pliable material and configured to provide power to the sensor.

11. The detector of claim 10, wherein the power pack is removably disposed on the pliable material.

12. The detector of claim 1, further comprising a wireless transmitter associated with the pliable material.

13. A system for detecting the integrity of a previously implanted prosthetic device comprising:
a detector including
a sensor configured to detect signals from the implant, and
securing means for securing the sensor in a sensing position relative to the implantable device;
a power source configured to provide power to the sensor; and
a computation means for processing data communicated by the detector and for displaying the processed data for diagnosing.

14. The system of claim 13, wherein the detector includes a wireless transmitter for transmitting information representative of the detected signals, and wherein the computation means includes a wireless receiver for receiving the transmitted information.

15. The system of claim 13, wherein the computation means is mobile and disposed on the detector.

16. The system of claim 13, wherein the power source is disposed on the detector.

17. The system of claim 13, wherein the securing means is a wrappable band.

18. The system of claim 13, including a stimulator for introducing mechanical loading to the device.

19. The system of claim 13, wherein the stimulator is one of a vibrator, an ultrasonic wave emitter, and both a vibrator and ultrasonic wave transmitter.

20. A method comprising:
disposing a sensor outside a patient's body for detecting a signal generated around an implanted prosthetic device;
detecting with the sensor the signal generated around the implanted prosthetic device; and
determining based on the detected signal the integrity of an interface between the previously implanted device and adjacent bone structure.

21. The method of claim 20, wherein the sensor is an acoustically sensitive sensor, and wherein detecting with the sensor includes detecting acoustics generated at the implant.

22. The method of claim 20, including recording data indicative of the detected signal.

23. The method of claim 20, including transmitting information indicative of the determined integrity of the interface.

24. The method of claim 20, wherein disposing a sensor includes wrapping a band around the patient's body in an area where the prosthetic device is implanted.

25. The method of claim 24, wherein disposing a sensor includes disposing multiple sensors in an array.

26. The method of claim 20, including introducing mechanical loading to the implanted prosthetic device.

27. The method of claim 26, wherein introducing mechanical loading includes vibrating tissue around the prosthetic device and emitting ultrasonic waves through tissue around the prosthetic device.

28. The method of claim 20, including wirelessly transmitting information indicative of the detected signal to a processing system.

29. A method comprising:
providing a sensor for disposal outside the patient's body, the sensor being an acoustically sensitive sensor for detecting an acoustical signal generated around an implanted prosthetic device; and
providing computer program product for formatting a computation means for determining based on the detected signal the integrity of an interface between the previously implanted device and adjacent bone structure.
